(19) United States
(12) Patent Application Publication
(10) Pub. No.: US 2017/0240948 A1
(43) Pub. Date: Aug. 24, 2017

(54) APPARATUS, SYSTEM AND METHOD FOR LIVE BACTERIA MICROSCOPY

(71) Applicant: TACOUNT EXACT LTD, Lower Galilee (IL)

(72) Inventors: VLADIMIR GLUKHMAN, Jerusalem (IL); Erez AZRAD, Lod (IL)

(73) Assignee: TACOUNT EXACT LTD, Lower Galilee (IL)

(21) Appl. No.: 15/115,248

(22) PCT Filed: Feb. 5, 2015
(86) PCT No.: PCT/IB2015/050892

§ 371 Date: Jul. 28, 2016

Related U.S. Application Data
(63) Continuation of application No. 14/271,190, filed on May 6, 2014, now abandoned.
(60) Provisional application No. 61/936,725, filed on Feb. 6, 2014.

(51) Int. Cl. C12Q 1/06; G02B 21/36; G02B 21/26; G02B 21/28; G01N 15/14; G01N 1/40

(52) U.S. Cl. CPC C12Q 1/06; G01N 15/1456; G01N 1/4077; G02B 21/26; G02B 21/28; G02B 21/362; G01N 2001/4088

(57) ABSTRACT

A system for detecting microbial cells in a sample includes an apparatus configured to image at least one cell in the sample, and a cooling device. The apparatus includes a holder having an internal portion and an external portion which are configured so as to secure a membrane between the portions, and an imaging device disposed above the external portion and configured to permit examination of the at least one cell. The apparatus further includes a stage attachable to a stage platform configured to connect to a motor, and a projecting member projecting from an upper surface of the stage and configured to receive and exchange solution. The cooling device includes a thermoelectric cooling element and/or at least one tube configured to circulate a cooling medium beneath the stage so as to cool the sample.

### CROSS-REFERENCE TO RELATED APPLICATIONS

The present application claims priority to U.S. Provisional Application No. 61/936,725, filed Feb. 6, 2014, and U.S. patent application Ser. No. 14/271,190, filed May 6, 2014. The disclosures of these prior applications are incorporated by reference herein in their entirety.

### FIELD OF THE INVENTION

The present invention relates to apparatus, systems and methods for live microscopy, and providing conditions that permit detection and evaluation of microorganisms under conditions that preserve metabolic activity. Relatedly, the apparatus, system and method is particularly relevant to the detection of viable culturable microorganisms in a sample that may contain viable non-culturable organisms, non-viable cells, and debris.

### BACKGROUND

Detection of disease-causing microorganisms in food and water is important for public health. The key indicator of disease risk is the number of organisms that can reproduce. The ability to reproduce is typically measured by the ability to grow in suitable culturable medium. The gold standard for counting the number of culturable microorganisms is plate counting. This method, however, is slow, expensive, and can be affected by aggregation and other problems. Plate counting also relies on the use of suitable medium on which the organism can grow.

Direct detection and enumeration of microorganisms in a sample of food or water, etc., would be desirable. Microscopy techniques allow for detection of microorganisms in samples of various substances. However, enumeration of the number of bacteria is a very unreliable proxy for the number of culturable organisms. This is because the population of microorganisms is not entirely or even mostly made of culturable organisms, but also includes dead cells, and viable non-culturable cells (hereinafter referred to as “VNC”). VNC are viable, but typically are less metabolically active than culturable cells. VNC may be the majority of viable cells in a population, and the proportion of viable cells that are VNC varies according to numerous conditions.

The presence of dead cells, VNC, and debris poses problems for detection and enumeration of culturable organisms. Dye-staining allows for detection of bacteria and differentiation between live and dead cells, but not between culturable cells and VNC.

WO 2010004567 A1 describes a method and kit for direct detection and enumeration of culturable microbial cells from a sample that may also contain VNC, dead cells, and debris. The method relies on differences between culturable, VNC, and dead cells in the rate at which signal emitting agents associate and disassociate with the membrane. Metabolically active, culturable, cells typically take up certain dyes at a faster rate than VNC or dead cells. The use of specific time points thereby permits distinction between culturable cells and all other cells and debris.

The method of WO 2010004567 is relatively fast, taking less than an hour. However, even several minutes under unfavourable conditions may alter the metabolic state of microorganisms. The present inventors therefore sought to develop an apparatus, and related systems and methods, that preserve the bacteria in a metabolically active state to permit accurate detection and enumeration.

### SUMMARY

In one embodiment, a system for detecting microbial cells in a sample includes an apparatus configured to image at least one cell in the sample. The apparatus comprises a holder comprising an internal portion and an external portion, the holder being configured to secure a member between the portions; an imaging device disposed above the external portion and configured to permit examination of the at least one cell; a stage attachable to a stage platform, the stage platform being configured to connect to a motor, and a projecting member projecting from an upper surface of the stage and configured to receive and provide solution. The system may further include a cooling device. The cooling device may comprise a thermoelectric cooling element disposed between the stage and the stage platform. Alternatively, cooling may be achieved by at least one inlet tube and at least one outlet tube configured to circulate a cooling medium beneath the stage, or by cooling device such as fans.

In another embodiment, a method for detecting microbial cells in a sample comprises filtering the sample through a membrane so as to capture a total number of cells in the sample on the membrane; applying the solution to the upper surface of the projecting member; fixing the holder to the projecting member such that the membrane is secured atop the projecting member contacts the solution and is supported by the upper surface of the projecting member; preparing the total number of cells for imaging by staining the total number of cells prior to cleansing, acquiring at least one image of the total number of cells in accordance with a sampling protocol, and detecting microbial cells.

In yet another embodiment, an apparatus for determining a number of culturable microbial cells in a sample comprises an internal ring and an external ring. The rings are configured so as to stretch a member between the rings and to maintain a position of the member relative to the rings. The apparatus further comprises an imaging device disposed above the external ring and configured to permit imaging of at least one cell in the sample; a stage attachable to a stage platform, the stage platform being configured to connect to a motor, and a projecting member projecting from an upper surface of the stage and configured to receive and provide solution, wherein a plurality of dimples provided on the stage are configured to engage with a plurality of pins on the stage platform.

### BRIEF DESCRIPTION OF THE DRAWINGS

The features, aspects and advantages of the present invention will become apparent from the following description, appended claims, and the accompanying exemplary embodiments shown in the drawings, which are briefly described below.

FIG. 1 is an exploded view of an apparatus for maintaining a suitable microbial environment while detecting culturable microbial cells in a sample, according to one embodiment.

FIG. 2 is a perspective view of a stage and a stage protrusion, according to one embodiment.

FIG. 3A is a perspective view of a stage platform, according to one embodiment.

FIG. 3B is a perspective view of an assembly of stage components, according to one embodiment.

FIG. 4A is a perspective view of a holder and a membrane, according to one embodiment.

FIG. 4B is an exploded view of a holder and a membrane, according to one embodiment.

FIG. 5A is an exploded view of a system for maintaining a suitable microbial environment while detecting culturable microbial in a sample, according to one embodiment.

FIG. 5B is an exploded view of the system shown in FIG. 5A, in which components within a stage platform are depicted.

FIG. 5C is a top view of a stage platform, according to one embodiment.

FIG. 6 is a flow diagram of a method for prolonged live cell imaging, according to one embodiment.

FIG. 7 is a flow diagram of a method for prolonged live cell imaging according to another embodiment.

FIGS. 8A-8D are images of bacteria obtained in accordance with one embodiment.

### DETAILED DESCRIPTION

The following detailed description is exemplary and explanatory only, and is not restrictive of the invention as claimed. In the following detailed description, reference is made to the accompanying drawings, in which similar symbols typically identify similar components, unless context dictates otherwise.

The drawings described above depict an apparatus configured to permit detection of culturable cells in a particular sample. Detection may further permit enumeration of the number of culturable cells. Techniques for determining the number of culturable cells in a sample containing, for example, culturable microbial cells, viable non-culturable microbial cells, and non-viable microbial cells are disclosed in U.S. patent application Ser. No. 13/003,128 to Glukhman, the entire contents of which are incorporated herein by reference for background information and the systems, kits, programs, processes and techniques disclosed therein.

Detection of the number of viable, culturable cells in a sample is challenging for various reasons. Notably, treatment protocols employed for imaging of the bacteria cells, and environmental conditions of the image acquisition process can cause stress to the imaged cells or even be lethal to them. In such circumstances, the cells can change their characteristics, for example, their metabolic behavior. Traditional imaging techniques suffer from difficulties in accurately capturing the characteristics of cells in their natural, intact state prior to preparation for imaging.

As a further complication, imaging generally requires sustaining a metabolic condition of the cells, as exhibited prior to testing, during the imaging process. Thus, the environment in which the cells are kept during image acquisition must be tailored so as to preserve a pre-test metabolic condition of the cells. The sampling process may require that the pre-test metabolic condition be maintained for half an hour or more than an hour, for example. Although imaging itself may take several minutes, other processes associated with sampling (e.g., preparation of the sample) take additional time. Accordingly, an appropriate environment must be maintained for the requisite time to ensure that the cells remain viable and in their pre-test metabolic condition during image acquisition. The embodiments described below allow for such pre-test metabolic conditions to be preserved.

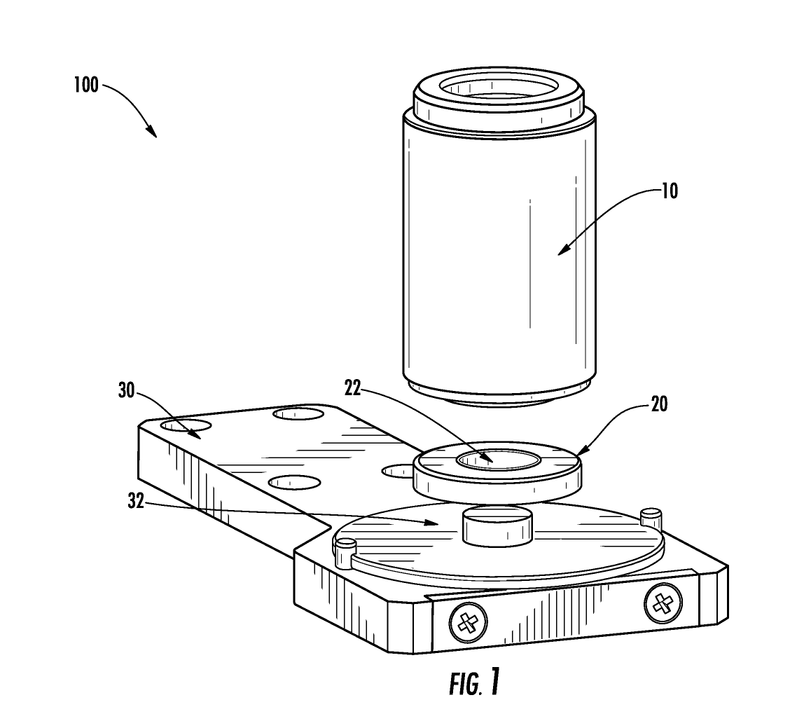

First, an overview of one embodiment of an apparatus for determining a number of culturable microbial cells in a sample will be described. FIG. 1 depicts an apparatus 100 comprising an imaging device having an objective lens 10, a holder 20, a stage platform 30, and a stage 32 disposed on the stage platform 30. Also depicted is a membrane 22 held in the holder 20. The apparatus 100 is configured to facilitate imaging of at least one cell in a sample, e.g., a sample of water or organic matter such as sewage or food.

The imaging device comprises components that allow for microscopic imaging of the sample. In addition to the objective lens 10 shown in FIG. 1, it is to be understood that the imaging device can include, in some embodiments, a plurality of lenses (e.g., an occular lens in addition to the objective lens 10) and additional components. For example, the additional components can include a turret or nose piece, a microscope frame, a microscope head, a diopter adjustment mechanism, an illumination source, and a mechanism to emit laser beams. In some embodiments, the turret is configured to revolve so as to move a plurality of objective lenses having varying powers of magnification.

More particularly, the imaging device allow for images to be taken of cells in the sample at the microscopic level. To this end, the cells in the sample are bound to a membrane 22. The membrane 22 is a membrane or filter. The holder 20 is configured to securely hold the membrane 22 in a fixed position in which the cells bound to the membrane are imaged by the imaging device.

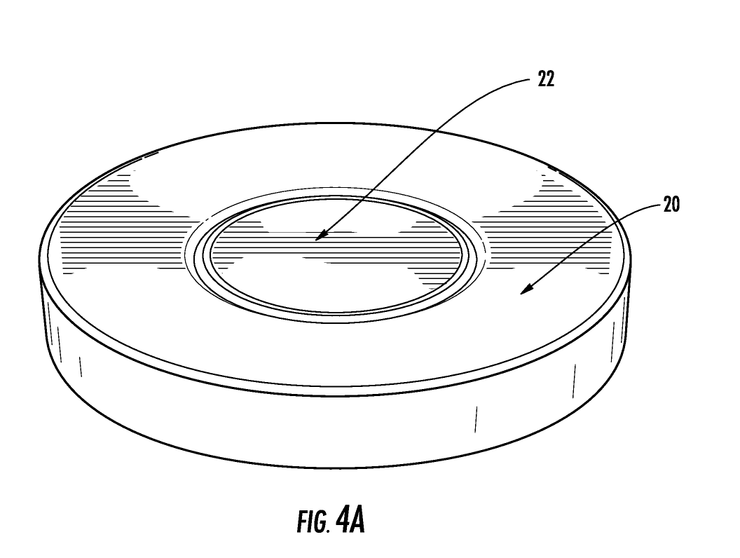

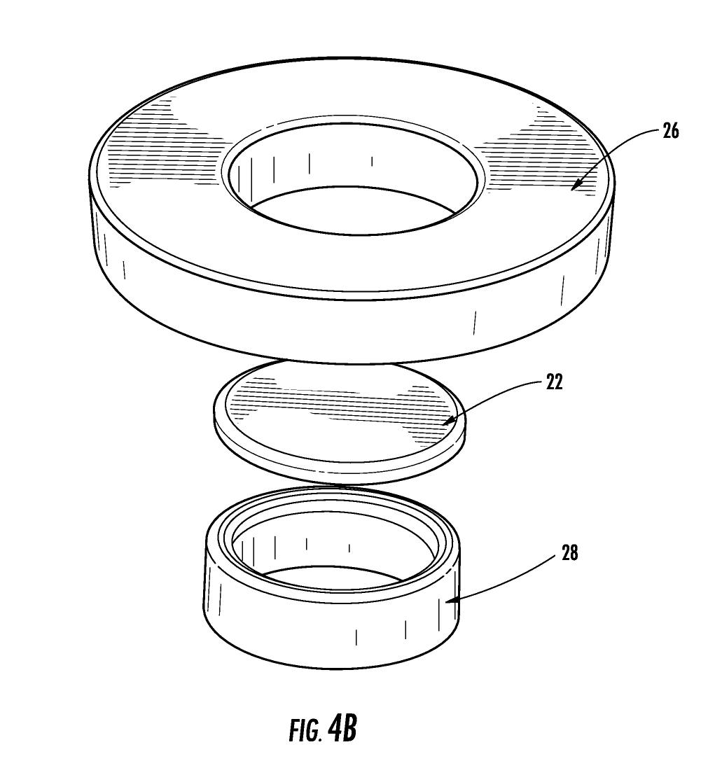

The holder 20, in some embodiments, comprises a plurality of rings, as will be described below. In other embodiments, the holder 20 is composed of rectilinear elements. In at least one embodiment, the holder 20 is a clamp. The disposition of the membrane 22 inside the holder 20 is discussed in more detail below in the context of FIG. 4B.

Referring again to FIG. 1, the stage 32 is positioned above the stage platform 30. The stage platform 30 provides a secure base for the stage 32. Although the stage 32 is depicted as a disc in the embodiment shown in FIG. 1, the stage 32 can have a variety of different shapes, e.g., a rectilinear shape or a flat shape with cut-outs. In some embodiments, the stage 32 is provided with clips.

The stage 32 is configured to be coupled to the stage platform 30 in a manner that permits movement of the stage 32 along a first axis and along a second axis. For example, with reference to a coordinate system as shown in the lower left corner of FIG. 1, the stage 32 is configured to move along an x-axis and a y-axis. The movement of the stage 32 along the x- and y-axes allows for samples to be delivered and positioned under the imaging device. More specifically, translational movement of the stage 32 along the x- and y-axes allows for repositioning of a given sample so that multiple fields of view can be obtained for the sample. Thus, the apparatus 100 provides for acquisition of numerous different images of the sample, improving the accuracy of the determination of the number of culturable cells detected by imaging.

Furthermore, although the stage 32 shown in FIG. 1 is configured to engage with the stage platform 30 via an engaging mechanism, as will be described below, in some embodiments, the stage platform 30 and the stage 32 are integrated together. In other words, the stage platform 30 and the stage 32 may be formed as a single part. In some embodiments, the stage 32 is a dais that is operated by a toggle or control lever to move along the x-, y-axes and z-axes. A controller, e.g., a microcontroller configured to carry out various operations relating to operation of the apparatus 100, can control the movement of the dais.

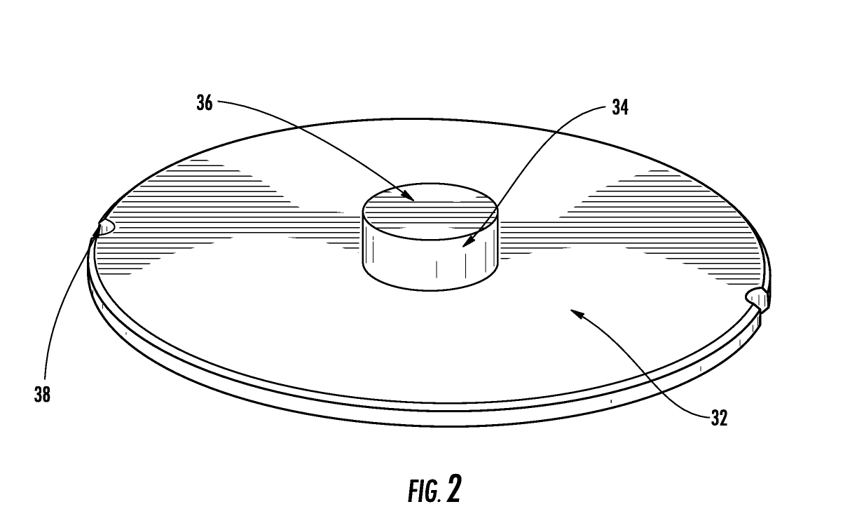

Turning now to FIG. 2, the stage 32 is depicted with engaging portions 38 and a projecting member 34. The projecting member 34 is a protrusion portion (a stage protrusion) that has an upper surface 36. Although FIG. 2 depicts the projecting member 34 as a cylinder centered on the stage 32, it will be understood that the projecting member 34 is not limited to this particular shape or orientation. For example, in some embodiments, the projecting member 34 juts from the stage 32 at a position other than the radial center. In some embodiments, the surface 36 is shaped as an ellipse or as a rectangle. In some embodiments, the stage platform 30, the stage 32, and the projecting member 34 are formed as a single component.

Further, in some embodiments, the stage platform 30 is configured to couple to and decouple from the imaging device. In some embodiments, the stage platform is configured to couple to and decouple from a motor (described below). In some embodiments, the stage platform 30 is configured to detach to the imaging device so as to extend from a first position to be in a second position. The second position, for example, may be beyond an enclosure of the imaging device, thus permitting the stage platform 30 to receive the membrane 22 and afterwards return to the first position. In some embodiments, the stage platform 30 is permanently fixed to the imaging device.

In some embodiments, the upper surface 36 of the projecting member 34 is configured to receive a solution, some amount of which is imparted to the membrane 22 when the membrane 22 contacts the projecting member 34. In some embodiments, the projecting member 34 comprises a solid cup-shaped member into which a sponge impregnated with the solution is inserted. In some embodiments, the projecting member 34 is provided with a tunnel or groove into which the solution is applied. When the projecting member 34 contacts the membrane 22, a quantity of the solution spreads to the membrane 22. In some embodiments, application of the solution comprises manually applying solution to the upper surface 36 of the projecting member 34. In some embodiments, application of the solution comprises directing the solution from a reservoir onto the upper surface 36 of the projecting member 34.

In certain embodiments, a fluid, for example, the aforementioned solution, can be exchanged so as to ensure that a fresh supply of fluid is provided to the membrane 22. The exchange of fluid can be accomplished via at least one microfluidic tube or at least one non-microfluidic tube. In some embodiments, at least one tube is provided to permit fluid to be exchanged on the upper surface 36 of the projecting member 34. In some embodiments, an exchange of fluid is accomplished via a plurality of tubes. In some embodiments, a groove, tube, tunnel, or channel is provided on the projecting member 34 so as to permit fluid exchange. Such configurations allow, in some embodiments, to permit fluid to be exchanged without a manual application of fluid on the membrane 22 or on the projecting member 34. In some embodiments, fluid exchange allows for staining and/or washing of cells on the membrane 22.

Further, in such embodiments, when the projecting member 34 contacts the holder 20, an amount of solution contacts the membrane 22. The solution received by the membrane 22 from the projecting member 34 serves to maintain the pre-test metabolic condition of the cells. The solution may be, for example, water, saline, a growth medium, or oil for maintaining an aerobic condition.

In some embodiments, an excess quantity of the solution is drained from the projecting member 34 and the membrane 22. For example, the projecting member 34 may include a socket or slit into which the excess quantity of the solution is discharged. By discharging the excess quantity of the solution, such embodiments can avoid distortion of the image caused by having a greater quantity of solution than the membrane 22 can receive.

Further, in some embodiments, the membrane 22 is completely immersed in the solution. In some embodiments, the membrane 22 is saturated by and immersed in an oil-based solution especially conductive to the growth of anaerobic bacteria. In some embodiments, the membrane 22 merely touches the solution without being immersed in the solution. In certain embodiments, the solution can diffuse gradually through the membrane 22. In other embodiments, the solution may rise up from the projecting member 34 so as to contact the membrane 22 via capillary action.

Referring again to FIG. 2, the engaging portions 38 of the stage 32 can be configured as recesses that form concavities in the stage 32. In some embodiments, the engaging portions 38 are attachment elements that are formed as dimples spaced at regular intervals along a periphery of the stage 32. In some embodiments, the engaging portions 38 are configured as latching or hooking members.

The components of the apparatus 100 (and a system 200 described below) may be made out of any suitable materials and structures. For example, in some embodiments, the holder 20 is made out of a durable plastic. In some embodiments, the projecting member 34 is composed of a rigid outer material and a porous spongiform inner material. In other embodiments, the projecting member 34 is substantially solid. Suitable materials include durable plastics, glass and metals that can be readily cleaned.

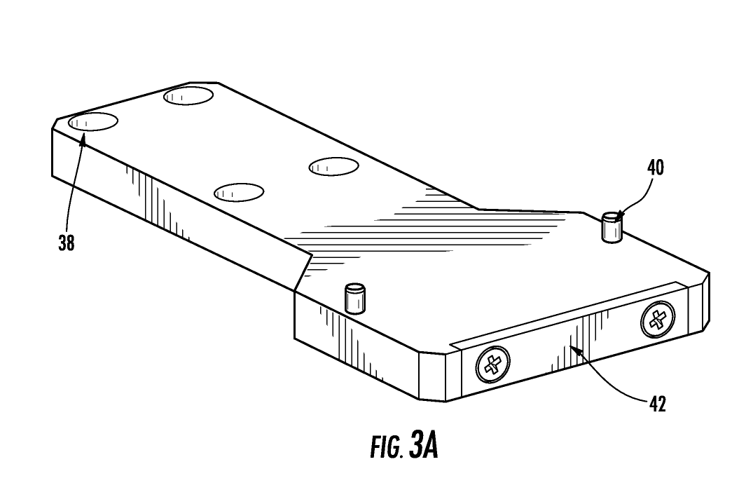

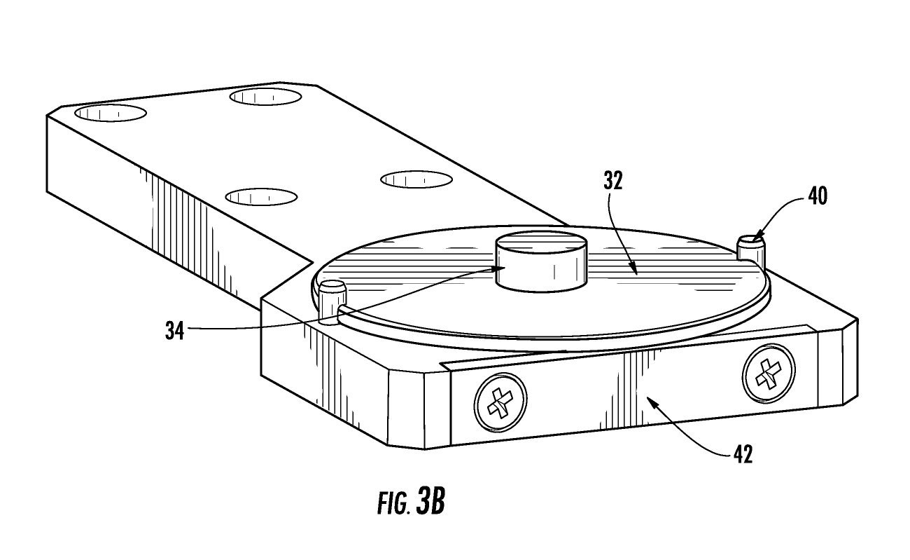

With reference to FIG. 3A, the stage platform 30 is shown as a base portion of the apparatus 100 having a neck portion and a front portion that is wider than the neck portion. The neck portion includes a plurality of apertures 38. The apertures 38 are configured so as to permit connection of the stage platform 30 to a motor (not shown). The motor can be a compact motor that allows for motorized control of a focusing mechanism of the imaging device and/or for movement of the stage 32. Such a motor can be provided with an encoded stepper drive that permits the motor to be repositioned repeatedly and accurately. Such a motor can be controlled, for example, via a software program configured to be executed in a processor of a computer and/or by a microcontroller for the motor. In some embodiments, the motor is connected to such a computer via a USB connection on the microcontroller for the motor.

Referring again to FIG. 3A, a plurality of pins 40 are depicted projecting from opposing sides of the stage platform 30. The pins 40 are attachment elements that are configured to come into abutting contact with the corresponding engaging portions 38 of the stage 32. The engaging portions 38 and the pins 40 are formed such that when the stage 32 is positioned such that the engaging portions 38 align with the pins 40, the pins 40 project into the concavities of the engaging portions 38. The pins 40 are thus snugly secured in position by the engaging portions 38. In some embodiments, the pins 40 are dowel pins. In some embodiments, the attachment elements of the stage platform 30 and the corresponding engaging portions of the stage 32 are configured to engage via a snap fit. In some embodiments, the engaging portions 38 are holes into which pegs on the stage 32 fit. In some embodiments, attachment elements 38 employ adhesive materials.

Still referring to FIG. 3A, a side of the stage platform 30 is provided with a magnetic element 42. The magnetic element 42 is configured such that when the apparatus 100 is positioned in an enclosure (not shown), the magnetic element 42 comes into contact with a door of the enclosure. For doors having magnetic portions or plates attached thereto, the magnetic element 42 is configured to keep the door securely shut by magnetic force. The enclosure, in some embodiments, is a microscope body to which the stage platform 30 can be secured by virtue of the magnetic element 42. In other embodiments, the magnetic element 42 allows for the stage platform 30 to be secured to the motor. The magnetic element is not provided in some embodiments.

Turning now to FIG. 4A, the holder 20 is shown with a membrane 22 that is a membrane or filter to which at least one cell is bound. The membrane 22 is positioned such that sides of the membrane 22 are held by the holder 20. The membrane 22 may be shaped as a thin disc that is stretched by the holder 20. In some embodiments, the stretching of the membrane 22 can result in the membrane 22 having sides that are folded and held by the holder 22. In some embodiments, the stretching of the membrane 22 does not result in the sides being folded but simply results in stretching. The holder 20 is configured to hold the membrane 22 such that, aside from any stretching of the membrane 22 by the holder 20 itself, no deformation of the membrane 22 occurs once the membrane 22 is held by the holder 20. The positioning of the membrane 22 in relation to elements of the holder 20 are shown in greater detail in FIG. 4B, as described below. In some embodiments, the membrane 22 is a flexible member that can be crimped, pinched, or cut.

Referring now to FIG. 4B, the holder 20 includes a first part 26 configurable as an external portion and a second part 28 configurable as an internal portion. The first and second parts 26, 28 are arranged such that the membrane 22 can fit between them. In some embodiments, the first part 26 has a larger diameter than both the membrane 22 and the second part 28, and the membrane 22 has a larger diameter than the second part 28. In such embodiments, therefore, a portion of the edges of the membrane 22 fold over or otherwise contact the sides of the second part 28 due to the force of the first part 26 exerted on the membrane 22. Although the first and second parts 26, 28 are shown as annular components in FIG. 4B, the holder 20 can be shaped differently in some embodiments and can fasten the membrane 22 into place without requiring the parts 26, 28 to be formed as separate components. An excess, folded portion of the membrane 22 may be trimmed or cut away, or left to protrude beyond the edges of the second part 28.

Referring once more to FIG. 4B, when the holder 22 is disassembled, the first part 26 and the second part 28 need not be in contact with each other. However, in some embodiments, the holder is assembled such that the second part 28 comes into contact with the first part 26 when the membrane 22 is positioned between the parts 26, 28. Once assembled, as shown in FIG. 4A, a top surface of the membrane 22 is on the same plane as a top surface of the first part 26 of the holding member 20. Thus, a top surface of the membrane 22 is exposed for imaging. However, in some embodiments, the top surface of the membrane is thereafter covered, for example, with a glass cover slip. Such a glass cover slip allows for an even flat surface to be provided with controllable refractive properties and reduced distortion. In some embodiments, an oil film is applied atop the glass cover slip. Providing such a glass cover slip can, in some embodiments, be particularly advantageous for imaging when there is a sample with a wet bottom surface (e.g., the wet bottom surface of the membrane 22 that has contacted solution).

Further, in some embodiments, the membrane 22 can be anchored to the projecting member 34 so as to be secured in place. The projecting member 34 thus provides a mechanical support to the membrane 22 so that deformation of the membrane 22 (beyond any deformation incurred in securing the membrane 22) can be avoided. By securing the membrane 22 in this manner, image acquisition can be carried out with high repeatability and improved focusing.

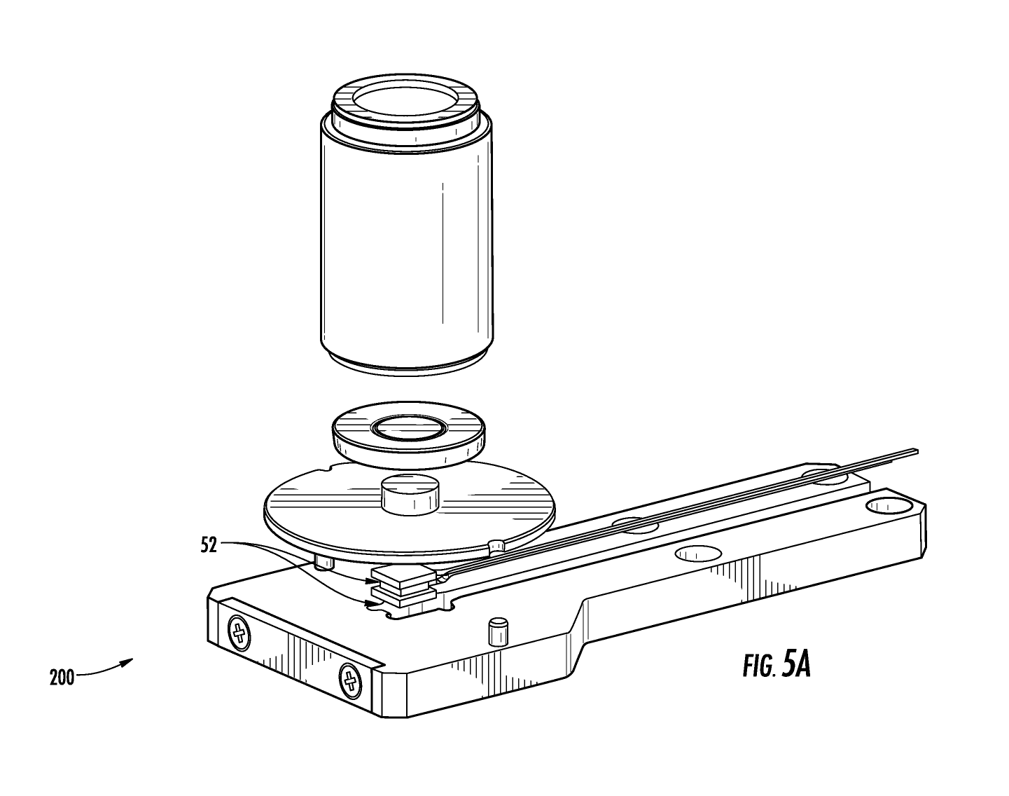

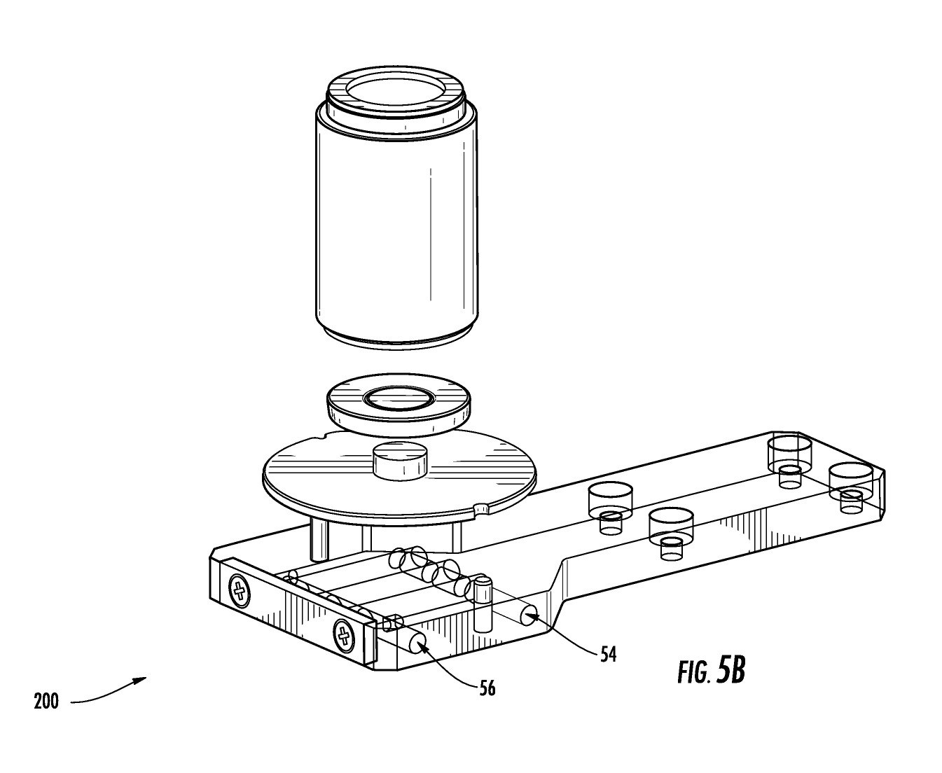

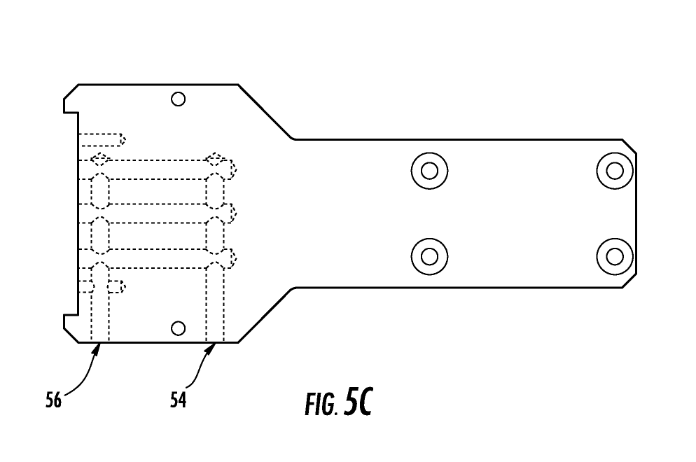

Referring now to FIG. 5A, a system 200 for determining a number of culturable microbial cells is depicted. The system 200 includes components that can be similar and/or analogous to the components of the apparatus 100 described above. Specifically, the system can include at least the imaging device having the objective lens 10, the holder 20, the stage platform 30, and the stage 32 disposed on the stage platform 30. In addition, the system 200 includes a cooling device. The cooling device can include, for example, a thermoelectric cooling element such as a Peltier module and/or a plurality of copper plates 52. The cooling device can also include, by way of further example, a fan or a plurality of fans. The plurality of copper plates 52 allow for improved thermal conductance. It should be understood that the cooling device does not require the plurality of copper plates 52 and that other elements may be present in the cooling device, as described below, for example, with respect to FIGS. 5B and 5C.

As shown in FIGS. 5B and 5C, the cooling device can include a plurality of tubes including an inlet tube 54 and an outlet tube 56. In some embodiments, a direction of flow within the tubes 54, 56 is parallel to the side of the stage platform 30 on which the magnetic element 42 is positioned. As shown in FIG. 5B, the inlet and outlet tubes 54, 56 are positioned within the stage platform 30 and beneath the stage 32. The inlet and outlet tubes 54, 56 are configured to circulate a coolant therein. For example, the inlet and outlet tubes 54, 56 are configured to circulate a cooling medium so as to cool the stage 32 and the cells bound to the membrane 22 secured in the holder 20. In some embodiments, the inlet and outlet tubes 54, 56 are configured to circulate solution. In some embodiments, the inlet and outlet tubes 54, 56 permit draining of an excess amount of solution out of the projecting member 34. The coolant can be, for example, a chemical coolant, water, or air. In related embodiments, the apparatus used for cooling can also be used for heating.

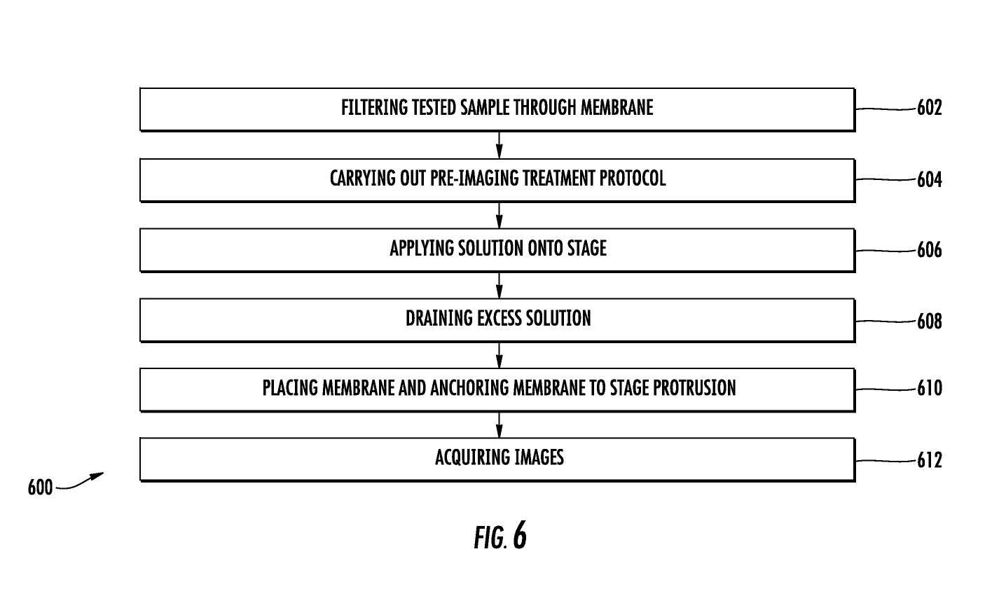

FIG. 6 depicts a flow diagram of a process 600 for prolonged live cell imaging. The process includes, at 602, filtering a tested sample through a membrane such as the membrane 22. In this manner, the cells to be imaged in an imaging device can be bound to the membrane 22. Next, at 604, a pre-imaging treatment protocol is carried out. For example, the bacteria cells can be dyed at 604, for example, with a fluorescent dye or other staining substance and can subsequently be washed. The dying and washing processes are illustrative of a preparatory regime before imaging, but other preparatory processes (and additional processes) may be carried out in accordance with a test protocol. The test protocol can be designed to achieve particular environmental and/or sampling conditions.

Next, at 606, the process includes applying a solution to an apparatus for imaging such as the apparatus 100 described above. More particularly, a solution can be applied to a top surface of a projecting member such as the projecting member 34 via a dropper, a reservoir, or other solution-delivering mechanisms. Following the application of solution at 606, an excess of solution can optionally be drained at 608.

The process 600 for prolonged live cell imaging includes arranging the membrane 22 with the holder 20. The arranging of the membrane 22 and the holder 20 may take place at the beginning of process 600, before the sample is filtered through the membrane. As described above, the membrane 22 can be stretched by the holder 20 so as to be substantially straight and not susceptible to further deformation. The holder 20 containing the membrane 22 is positioned with the projecting member 34. More particularly, the membrane 22 is positioned such that the solution previously applied on the projecting member 34 contacts the membrane 22. Furthermore, at 610, the projecting member 34 is disposed so as to mechanically support the membrane 22 and to be anchored to it. In this manner, the projecting member 34 can protect the membrane 34 from deformation. Following anchoring of the membrane 22 to the projecting member 34, images of the cells can be carried out at 612.

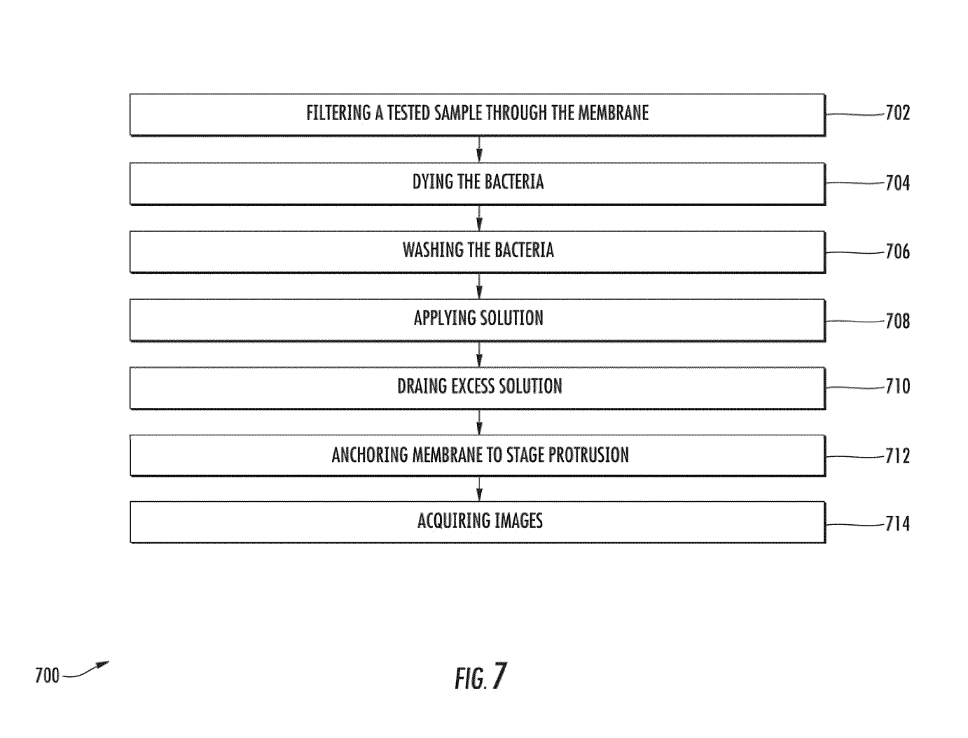

FIG. 7 depicts a flow diagram of a process 700 for prolonged live cell imaging. The process includes, at 702, filtering a tested sample through a membrane such as the membrane 22 following arrangement of the membrane 22 with the holder 20. In this manner, the cells to be imaged in an imaging device can be bound to the membrane 22. Next, at 704 and 706, a preparatory protocol can be carried out. For example, the bacteria cells can be dyed at 704, for example, with a fluorescent dye or other staining substance. Further, at step 706, the cells can thereafter be washed.

Referring again to FIG. 7, following treatment in accordance with the protocol, solution is applied to an apparatus for imaging such as the apparatus 100 described above, as indicated at 708. As with the process 600 at 606, a solution can be applied to a top surface of a projecting member (such as the projecting member 34) via a dropper, a reservoir, or other solution-delivering mechanisms. Following the application of solution at 708, an excess of solution can optionally be drained at 710. To this effect, a draining structure can be provided for the projecting member 34 to facilitate discharge of excess solution. Alternatively, in some embodiments, a vacuum may suction out excess solution. The vacuum, in some embodiments, is disposed beneath the projecting member 34.

Once more referring to FIG. 7, following the application of solution at 708 and the optional drainage of an excess amount of such solution at 710, the membrane 22 is anchored to the projecting member 34 at 712. Following anchoring of the membrane 22 to the projecting member 34, images of the cells can be carried out at 714.

A process for determining a number of culturable microbial cells according to one embodiment is described below. The process begins with (1) arranging a membrane in a membrane holder, (2) filtering a tested sample through the membrane as to capture all the bacteria from the tested sample onto the filter. The process further involves (3) treating the bacteria according to the desired test protocol, such as dying the bacteria with a fluorescent dye, washing the cells or other preparatory processes that may be carried out so as to obtain desirable environmental and/or sampling conditions, for example. Next, the process involves (4) applying an appropriate solution to the upper surface of the stage protrusion, either by manually placing a drop, or by using a reservoir of solution, and (5) optionally draining excess solution to prevent image distortion. The process further entails (6) placing the filter on the stage protrusion such that the filter holder is anchored to the protrusion. The filter is placed to be in close proximity to the upper surface of the protrusion, and the filter is in touch with the solution and mechanically supported by the upper surface of the stage protrusion so as to prevent filter deformation and to enhance focus. The process further includes (7) acquiring images according to a desirable protocol.

It should be noted that the sequence of processes illustrated in FIGS. 6 and 7 are not limited to any particular sequence, and that some embodiments may omit particular processes or include additional processes as appropriate. For example, in some embodiments, following a first image acquisition, a validation process is performed involving plate counting so as enable comparison with the results of the image acquisition. Alternative processes are discussed below by way of illustration.

An alternative process of some embodiments includes, for example, binding cells from a sample to a membrane or filter such as the membrane 22, fixing the membrane 22 to a holder such as the holder 20, and applying solution to a stage protrusion such as the protrusion 34. The process can further include draining an excess amount of the solution applied to the stage protrusion before arranging the membrane 22 and the holder 20 on the stage protrusion 34.

In some embodiments, by way of example, the method can further include providing a glass cover slip atop the membrane 22 after the membrane 22 is secured in the holder 20. In some embodiments, the method can include staining the cells bound to the membrane 22 prior to fixing the membrane 22 to the holder 20. In some embodiments, the method includes mounting the membrane 22 to the holder 20, and subsequently staining cells bound to the membrane 22. In some embodiments, the cells are bound to the membrane 22, which is then fixed to the holder 20 and positioned to contact the projecting member 34 prior to staining.

In some embodiments, following arrangement of the membrane 22 and the holder 20, the cells on the membrane 22 are suspended with a substance such as a fluorescent dye, for example. After the staining, the cells are washed so as to reduce an amount of dye present in a background image to be taken of the dyed cells. Once the cells are washed, the cells are mounted to a stage such as the stage 32. It should be noted that the suspending and washing processes are illustrative of an exemplary preparatory process prior to imaging of the cells and may be omitted in some embodiments. Other preparatory processes may be carried out so as to obtain desirable environmental and/or sampling conditions for the cell to be imaged by the imaging device.

In some embodiments, the washing and staining of microbial cells can be performed before the cells are bound to the membrane, or can be performed on the membrane. The staining on the membrane can be before or after mounting the membrane 22 in the holder 20. In some embodiments, the membrane is assembled prior to filtering because the assembled membrane fits the filtering machine and thus goes directly from filtering to staining and imaging. When washing and staining is performed after mounting the member in the holder, the remaining features of the apparatus are suitably configured for the exchange of solutions with the membrane, such as by provision of tubes to provide for the exchange of solutions. Performing the staining and washing on a membrane 22 in the holder 20 may be an element of an automated process, e.g. to examine multiple samples.

In some embodiments, once the cells are mounted to the stage, the cells can undergo imaging by the imaging device. The imaging device thus allows images of the cells to be acquired. In some embodiments, the imaging device allows prolonged imaging of live cells so as to keep the cells alive and metabolically intact during imaging. For example, in some embodiments, live cells are imaged so as to determine the quantity of culturable cells from samples including dead cells and VNCs. Such embodiments can differentiate between viable culturable cells (VCC) and VNCs.

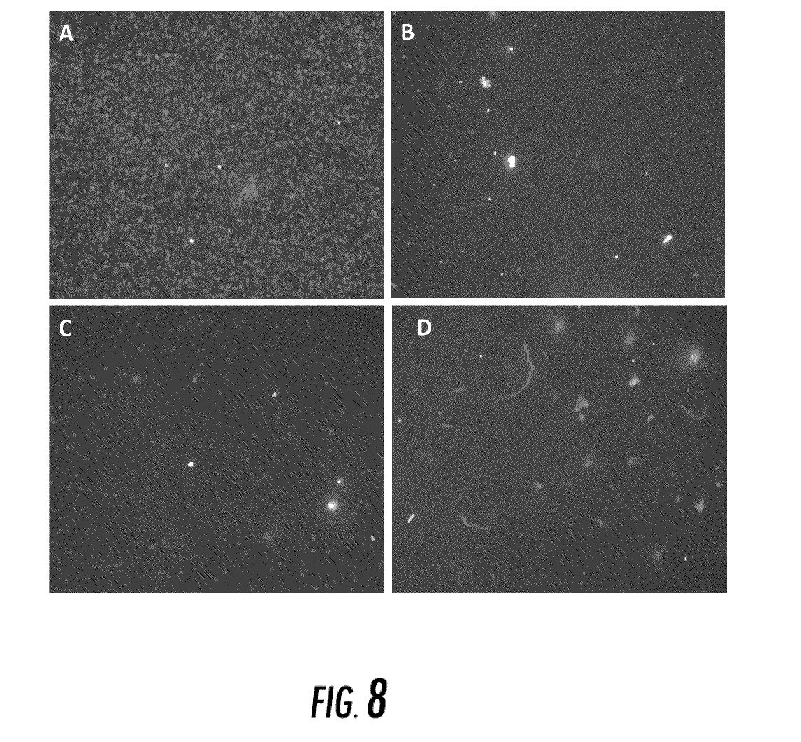

FIGS. 8A-8D depict images of membranes obtained in accordance with the above-described embodiments. FIGS. 8A and 8C are images obtained for membranes imaged with a drop of liquid placed on the top of the stage prior to the membranes being placed on the stage. FIGS. 8B and 8D are images of membranes placed directly atop the top surface of the stage, without liquid. Other than the presence of a drop of solution on the stage, FIGS. 8A and 8B were the same bacterial sample treated the same way. Likewise, FIGS. 8C and 8D were the same sample treated the same way, except for the presence of a drop of solution. Comparing the A/B and C/D pairs therefore demonstrates how drying for only a few minutes can have a significant effect on the image and the ultimate number of VCCs that are counted.

To obtain the images of FIGS. 8A-8D, bacteria from calibrated bacterial stocks were collected on the membranes. The membranes were suspended in a dye solution for a predetermined amount of time. The membranes were washed in PBS, and then the membranes were placed on the stage apparatus (with or without liquid).

In particular, FIG. 8A depicts a membrane suspended in a dye solution for 3.5 minutes for which a drop of liquid was placed on the top surface placed on the stage prior to the membrane being placed on the stage. FIG. 8B depicts a membrane suspended in a dye solution for 3.5 minutes and placed directly on the top surface of the stage without liquid being added to the top surface of the stage. FIGS. 8A and 8B are images taken of a calibrated total bacterial mix from mineral water (HPC count 8,500 CFU/ml). The images were taken after 3.5 minutes using green illumination.

In contrast to FIGS. 8A and 8B, FIGS. 8C and 8D are images taken after 6.5 minutes using UV illumination for a calibrated E. coli culture (HPC count 5,580 CFU/ml). In particular, FIG. 8C depicts a membrane suspended in a dye solution for 6.5 minutes for which a drop of liquid was placed on the top surface placed on the stage prior to the membrane being placed on the stage. FIG. 8D depicts a membrane suspended in a dye solution for 6.5 minutes and placed directly on the top surface of the stage without liquid being added to the top surface of the stage. Table 1 below summarizes the results for the membranes shown in FIGS. 8A-8D.

TABLE 1

| | A | B | | |
|---|---|---|---|---|
| | Wet | Dry | C | D |
| | membrane | membrane | Wet | Dry |
| | Total | Total | membrane | membrane |
| | bacteria mix | bacteria mix | E. Coli | E. Coli |
| | After | After | After | After |
| | 3.5 minutes | 3.5 minutes | 6.5 minutes | 6.5 minutes |
| HPC calibrated | 8,500 | 8,500 | 5,580 | 5,580 |
| stock solution | | | | |
| Microscopic | 8,412 | 4,561 | 5,630 | 0 |
| count - per | | | | |
| embodiments, | | | | |
| standardized | | | | |
| count | | | | |

The results shown in Table 1 demonstrate that without maintaining the membranes in a wet state, the membranes dry very fast and the cells change their metabolic state, and may lose their culturability, dry out and die. FIG. 8B shows a dramatic decrease in the VCC count compared to FIG. 8A (4,561 VCC/ml in FIG. 8B compared to 8.412 VCC/ml in FIG. 8A), after 3.5 minutes. On the other hand, when a drop of water is placed on the top surface of the stage, and the membrane is therefore kept wet, the cells not only stay alive but are also metabolically intact, such that the culturable cell count even after 3.5 minutes is still comparable to the CFU of the same culture (calibrated culture, where the count should be normalized). As shown in FIG. 8D, after 6.5 minutes, the membrane dried out completely and no culturable cells were detected on the membrane. In contrast, the membrane shown in FIG. 8C was maintained wet, and the cells were metabolically intact, having a VCC count comparable to the CFU count of the same culture.

Thus, the present device, composition and related method permit accurate enumeration of VCC.

The present invention is not limited to the above-described preferred embodiments. Various alternatives, modifications and equivalents may be used. Therefore, the above embodiments should not be taken as limiting the scope of the invention.

1. A system for prolonged real-time imaging of single cell live bacteria, the system comprising:
a holder comprising an internal portion and an external portion, the holder being configured to secure a membrane between the portions,
an imaging device disposed above the external portion and configured to permit examination of at least one cell, and
a stage attachable to a stage platform, the stage platform being configured to connect to a motor.

2. The system of claim 1, further comprising a projecting member projecting from an upper surface of the stage and configured to receive and provide solution.

3. The system of claim 1, further comprising a cooling device including a thermoelectric cooling element disposed between the stage and the stage platform.

4. The system of claim 1, further comprising a cooling device including at least one inlet tube and at least one outlet tube configured to circulate a cooling medium beneath the stage so as to cool a sample.

5. A system for detecting microbial cells in a sample, the system comprising:
an apparatus configured to image at least one cell in the sample, the apparatus comprising
a holder comprising an internal portion and an external portion, the holder being configured to secure a membrane between the portions,
an imaging device disposed above the external portion and configured to permit examination of the at least one cell;
a stage attachable to a stage platform, the stage platform being configured to connect to a motor, and
a projecting member projecting from an upper surface of the stage and configured to receive and provide solution; and
a cooling device comprising at least one of
a thermoelectric cooling element disposed between the stage and the stage platform, and
at least one inlet tube and at least one outlet tube configured to circulate a cooling medium beneath the stage so as to cool the sample.

6. The system of claim 5, wherein
the internal portion is a first ring, and the external portion is a second ring, and
a diameter of the first ring is less than a diameter of the second ring.

7. The system of claim 1, wherein the stage comprises a first plurality of attachment elements to facilitate coupling to the stage platform.

8. The system of claim 7, wherein the first plurality of attachment elements comprises recesses configured to align with a corresponding second plurality of attachment elements disposed on the stage platform so as to lock the stage to the stage platform.

9. The system of claim 8, wherein the second plurality of attachment elements comprises pins projecting from the stage platform.

10. The system of claim 1, wherein a magnetic member is provided on one side of the stage platform.

11. The system of claim 1, wherein the stage platform comprises a plurality of apertures configured to permit connection to the motor.

12. The system of claim 5, wherein the at least one inlet tube and the at least one outlet tube are disposed such that an outlet port of the outlet tube parallels an inlet port of the inlet tube.

13. The system of claim 3, wherein the thermoelectric cooling element is a Peltier module.

14. The system of claim 3, wherein the cooling device further comprises a plurality of copper plates.

15. The system of claim 2, wherein the projecting member comprises a drain portion configured to permit drainage of an excess amount of the solution therethrough.

16. The system of claim 2, wherein the projecting member comprises a tube to permit an exchange of fluid on the surface of the projecting member.

17. The system of claim 2, wherein the projecting member is integrated with the stage.

18. A method for detecting microbial cells in a sample, the method comprising:
filtering the sample through a membrane so as to capture a total number of cells in the sample on the membrane;
treating the cells according to a test protocol;
applying solution to an upper surface of a projecting member of a stage;
fixing the membrane to the projecting member of the stage by securing a holder for the membrane to the projecting member such that the membrane contacts the solution and is supported by the upper surface of the projecting member;
acquiring at least one image of the total number of cells in accordance with the test protocol, and
detecting microbial cells.

19. The method of claim 18, further comprising:
draining an excess amount of the solution from the upper surface of the projecting member so as to prevent distortion.

20. The method of claim 18, further comprising
washing the cells prior to the application of the solution.

21. The method of claim 18, wherein the sampling protocol comprises dying the cells of the sample with a fluorescent dye.

22. The method of claim 18, wherein the application of the solution comprises manually applying solution to the upper surface of the projecting member.

23. The method of claim 18, wherein the application of the solution comprises directing the solution from a reservoir onto the upper surface of the projecting member.

24. An apparatus for determining a number of culturable microbial cells in a sample, the apparatus comprising:
an internal ring and an external ring, the rings being configured so as to stretch a membrane between the rings and to maintain a position of the membrane relative to the rings;
an imaging device disposed above the external ring and configured to permit imaging of at least one cell in the sample;
a stage attachable to a stage platform, the stage platform being configured to connect to a motor, and
a projecting member projecting from an upper surface of the stage and configured to receive and provide solution,
wherein a plurality of dimples provided on the stage are configured to engage with a plurality of pins on the stage platform.

25. The apparatus of claim 24, further comprising:
at least one conduit configured to permit exchange of the solution.

26. The apparatus of claim 25, wherein the at least one conduit comprises a first conduit configured to intake the solution, and a second conduit configured to discharge the solution.

27. The apparatus of claim 24, wherein the at least one conduit is a spongiform conduit provided in a center of the projecting member.
